(19) United States
(12) Patent Application Publication
(10) Pub. No.: US 2010/0249576 A1
(43) Pub. Date: Sep. 30, 2010

(54) DEVICES, SYSTEMS, AND METHODS OF TRACKING ANATOMICAL FEATURES

(75) Inventors: Mohsen Askarinya, Chandler, AZ (US); Richard Lee Brown, Mesa, AZ (US)

Correspondence Address: MEDTRONIC;Attn: Noreen Johnson - IP Legal Department, 2600 Sofamor Danek Drive, MEMPHIS, TN, 38132, US

(73) Assignee: WARSAW ORTHOPEDIC, INC., An Indiana Corporation, Warsaw, IN (US)

(21) Appl. No.: 12/413,112

(22) Filed: Mar. 27, 2009

(51) Int. Cl. A61B 5/05

(52) U.S. Cl. 600/424

(57) ABSTRACT

Devices, systems, and methods of tracking one or more anatomical features of a patient are disclosed. In one embodiment, a method of tracking anatomical features of a patient includes introducing a first implantable magnetic source into a patient's body, fixedly securing the magnetic source to a first anatomical feature, and monitoring the magnetic field generated by the magnetic source with a magnetic sensing system positioned outside the patient's body in order to track a position of the magnetic source. In another embodiment, a method of tracking anatomical features of a patient includes introducing a first implantable magnetic sensor into a patient's body, fixedly securing the sensor to a first anatomical feature of the patient, and monitoring a strength of a magnetic field generated by a magnetic source positioned outside the patient's body with the sensor in order to track a position of the sensor relative to the magnetic source.

### BACKGROUND

There exists a need in the medical field to allow healthcare providers the ability to safely and effectively monitor the position and movement of patients' internal anatomical structures. Current methods for monitoring the position and movement of anatomical structures include the use of radiographic imaging (i.e. x-rays) and magnetic resonance imaging devices. However, the expense associated with these methods for patients and/or healthcare providers can prohibit access to these traditional methods. Further, patients and healthcare providers can be unnecessarily exposed to dangerous cumulative levels of radiation through the use of the devices and systems associated with the traditional methods. Accordingly, minimally invasive devices, systems, and methods for monitoring the position and movement of anatomical structures of patients that avoid the problems associated with legacy medical techniques are needed.

### SUMMARY

Disclosed herein are devices, systems, and methods that enable a healthcare provider to monitor the position and/or movement of anatomical structures within a patient's body. In one embodiment, the system comprises a sensor, a magnetic source, and a receiver. In some instances, the sensor is a device capable of detecting and measuring an electromagnetic field. The magnetic source is a source capable of producing an electromagnetic field, including both permanent magnets and electronic circuits. In one embodiment, the sensor is attached to an anatomical structure inside a patient's body while the magnetic source is located outside of the patient's body. In that regard, a magnitude of the magnetic field is measured by the sensor positioned within the body and communicated to the receiver. The measured changes in the magnitude of the magnetic field are utilized to track movement of the sensor and, thereby, the anatomical structure to which the sensor is attached. In some instances, the receiver is a device that is capable of communicating with the sensor. In some embodiments, the receiver is configured to communicate wirelessly with the sensor in order to receive information relating to magnitude or strength of the magnetic field being measured by the sensor. In some instances the receiver is located outside of the patient's body. In other embodiments the receiver is located inside of the patient's body as well. In one particular embodiment, the receiver is integrated with the sensor such that the sensor functions as both the sensor and the receiver. In other embodiments, the receiver is spaced from the sensor within the patient's body.

Alternatively, in another embodiment the magnetic source is attached to an anatomical structure inside a patient's body while the sensor is located outside of the patient's body. In that regard, a magnitude of the magnetic field generated by the magnetic source is measured by the sensor positioned outside the body. The measured changes in the magnitude of the magnetic field are utilized to track movement of the magnetic source and, thereby, the anatomical structure to which the magnetic source is attached. In some instances where the magnetic source is positioned within the patient's body, the sensor and the receiver are joined together to form a single external component. Further, in some instances the sensor and/or the receiver includes a processor programmed to calculate the relative movement and/or position of the magnetic source based on the detected changes in the magnitude of the magnetic field.

In operation, an implantable version of either the magnetic source or the sensor is attached to an anatomical structure while the other is positioned outside of the patient's body. In some instances, the magnetic source is configured to generate an electromagnetic field of known strength. The sensor detects and measures the strength of the electromagnetic field generated by the magnetic source. Therefore, changes in the absolute strength of the electromagnetic field as detected by the sensor are used to determine a position of the sensor relative to the magnetic source. In some instances, the system is calibrated in order to define an origin or zero point from which the movement of the anatomical feature is tracked. In that regard, in some instances the sensor is calibrated. Specifically, the sensor is calibrated such that a baseline strength for the electromagnetic field generated by the magnetic source is determined at certain distance between the sensor and the magnetic source. The sensor is then able to monitor changes in the strength of the electromagnetic field with respect to the baseline measurement. Relative changes in the strength of the measured electromagnetic field to the baseline measurement are utilized to determine the relative movement between the sensor and the magnetic source. In other instances, the magnetic source is calibrated. For example, in some instances the magnetic source is capable of generating magnetic fields of varying strength. Accordingly, with the sensor positioned at a known distance and/or orientation relative to the magnetic source the strength of the magnetic source is adjusted to a desired baseline level for the known distance and/or orientation. Subsequently, changes in the strength of the measured electromagnetic field to the baseline level are utilized to determine the relative movement between the sensor and the magnetic source. Thus, in this manner the position of the anatomical structure is monitored by measuring the changes in the magnetic field as measured by the sensor.

Devices, systems, and methods of tracking one or more anatomical features of a patient are disclosed.

In one embodiment, an implantable magnetic source is disclosed. The implantable magnetic source includes a permanent magnet in some instances. The implantable magnetic source includes circuitry for generating a magnetic field in some instances. In some embodiments, the implantable magnetic source includes a housing configured for engaging bony anatomical features. In some embodiments, the implantable magnetic source includes a housing configured to be secured to soft tissue anatomical features.

In another embodiment, an implantable sensor is disclosed. The implantable sensor includes a magnetometer in some instances. In some embodiments, the implantable sensor includes a housing configured for engaging bony anatomical features. In some embodiments, the implantable sensor includes a housing configured to be secured to soft tissue anatomical features.

In one embodiment, a method of tracking anatomical features of a patient is disclosed. The method includes introducing a first implantable magnetic source into a patient's body, fixedly securing the magnetic source to a first anatomical feature, and monitoring the magnetic field generated by the magnetic source with a magnetic sensing system positioned outside the patient's body in order to track a position of the magnetic source. In some instances, the method further comprises second and third implantable magnetic sources into the patient's body, fixedly securing them to second and third portions of the first anatomical structure, and monitoring the magnetic fields generated by the first, second, and third implantable magnetic sources with the magnetic sensing system positioned outside the patient's body in order to track positions of the first, second, and third implantable magnetic sources. In some instances, the method further comprises calibrating the magnetic sensing system positioned outside the body for use with the first, second, and third implantable magnetic sources. In some instances, the first anatomical feature is a vertebra and the first portion of the vertebra is a first transverse process, the second portion of the vertebra is a second transverse process, and the third portion of the vertebra is a spinous process. In some embodiments, the implantable magnetic source is a permanent magnet. In some embodiments, the implantable magnetic source is an electrical circuit configured to generate a magnetic field. In that regard, in some instances the electrical circuit is configured to generate magnetic fields of varying strength.

In another embodiment, a method of tracking anatomical features of a patient includes introducing a first implantable magnetic sensor into a patient's body, fixedly securing the sensor to a first anatomical feature of the patient, and monitoring a strength of a magnetic field generated by a magnetic source positioned outside the patient's body with the sensor in order to track a position of the sensor relative to the magnetic source. In some instances, the method further comprises introducing second and third implantable magnetic sensors into the patient's body, fixedly securing the second and third implantable magnetic sensors to the first anatomical feature, and monitoring the strength of the magnetic field generated by the magnetic source positioned outside the patient's body with the first, second, and third implantable magnetic sensors in order to track positions of the first, second, and third implantable magnetic sensors relative to the magnetic source. In some instances, the method includes calibrating the first, second, and third implantable magnetic sensors for use with the magnetic source. In some embodiments, calibrating the first, second, and third implantable magnetic sensors comprises obtaining a baseline measurement of the strength of the magnetic field generated by the magnetic source at a baseline position. In some instances, monitoring the strength of the magnetic field generated by the magnetic source in order to track positions of the first, second, and third implantable magnetic sensors comprises determining a relative position of the first, second, and third implantable magnetic sensors to the baseline position.

In another embodiment, a method of the present disclosure includes gaining minimally invasive access to a first bony anatomical feature, introducing at least one implantable device containing a permanent magnet into a patient's body through the minimally invasive access, threadingly engaging a housing of the at least one implantable device with the first bony anatomical feature through the minimally invasive access, closing the minimally invasive access to the first bony anatomical feature with the at least one implantable device threadingly engaged with the first bony anatomical feature, and monitoring a magnetic field generated by the permanent magnet contained by the at least one implantable device with a magnetic sensing system positioned outside the patient's body in order to track a position of the at least one implantable device threadingly engaged with the first bony anatomical feature.

No limitation to the scope of the present disclosure should be implied based on the present summary. In that regard, additional details, features, and aspects of the present disclosure will be apparent to those skilled in the art based on the following detailed description and the corresponding drawing figures.

### BRIEF DESCRIPTION OF THE DRAWINGS

In the accompanying drawings, which are incorporated in and constitute a part of this specification, embodiments of the present disclosure are illustrated, which, together with the description provided herein, serve to exemplify aspects of the present disclosure.

FIG. 1 is a schematic diagram of a system for monitoring the position and movement of anatomical structures according to one embodiment of the present disclosure.

FIG. 2 is a flowchart of a method of affixing a magnetic source to an anatomical structure and monitoring the position and movement of the anatomical structure according to one embodiment of the present disclosure.

FIG. 3 is a front view of a patient whose anatomical features are being monitored with a system for monitoring the position and movement of anatomical structures, incorporating aspects of the system shown in FIG. 1 and the method shown in FIG. 2.

FIG. 4 is a schematic diagram of an external sensor according to one embodiment of the present disclosure.

FIG. 5 is a flowchart of a method for affixing a magnetic source to a vertebra of a spinal column to monitor the position and movement of the vertebra according to one embodiment of the present disclosure.

FIG. 6 is a partial cross-sectional, perspective view of a magnetic source being implanted adjacent a vertebra of a spinal column of a patient according to one aspect of the present disclosure.

FIG. 7 is a partial cross-sectional, front view similar that of FIG. 6, but showing the magnetic source engaged with the vertebra of the spinal column of the patient.

FIG. 8 is a side view of a system monitoring the position and movement of vertebrae of a patient according to one embodiment of the present disclosure.

FIG. 9 is a side view of a bone screw having a magnetic source disposed therein according to one embodiment of the present disclosure.

FIG. 10 is a perspective view of the bone screw of FIG. 9 with an outer housing of the bone screw shown in phantom.

FIG. 11 is a side view of the magnetic source disposed within the bone screw of FIGS. 9 and 10.

FIG. 12 is an end view of a bone penetrating portion of the bone screw of FIGS. 9 and 10.

FIG. 13 is an end view of a head portion of the bone screw of FIGS. 9, 10, and 12, according to one embodiment of the present disclosure.

FIG. 14 is an end view of a head portion of the bone screw of FIGS. 9, 10, and 12, according to another embodiment of the present disclosure.

FIG. 15 is a schematic diagram of a system for monitoring the position and movement of anatomical structures according to another embodiment of the present disclosure.

FIG. 16 is a flowchart of a method of affixing a sensor to an anatomical structure and monitoring the position and movement of the anatomical structure according to one embodiment of the present disclosure.

FIG. 17 is a front view of a patient whose anatomical features are being monitored with a system for monitoring the position and movement of anatomical structures, incorporating aspects of the system shown in FIG. 15 and the method shown in FIG. 16.

FIG. 18 is a flowchart of a method for affixing a sensor to a vertebra of a spinal column to monitor the position and movement of the vertebra according to one embodiment of the present disclosure.

FIG. 19 is a partial cross-sectional, front view of a sensor being implanted adjacent a vertebra of a spinal column of a patient according to one aspect of the present disclosure.

FIG. 20 is a partial cross-sectional, front view similar that of FIG. 19, but showing the sensor engaged with the vertebra of the spinal column of the patient.

FIG. 21 is a side view of a system monitoring the position and movement of vertebrae of a patient according to another embodiment of the present disclosure

FIG. 22 is a perspective view of a bone screw according to another embodiment of the present disclosure.

FIG. 23 is a perspective view of the bone screw of FIG. 22 with an outer housing of the bone screw shown in phantom.

FIG. 24 is a cross-sectional side view of the bone screw of FIGS. 22 and 23.

FIG. 25 is an end view of a head portion of the bone screw of FIGS. 22, 23, and 24, according to one embodiment of the present disclosure.

FIG. 26 is a schematic diagram of a receiver according to one embodiment of the present disclosure.

### DETAILED DESCRIPTION

The present disclosure relates generally to systems and methods for monitoring the position and movement of anatomical structures within the human body. For the purposes of promoting an understanding of the principles of the present disclosure, reference will now be made to embodiments or examples illustrated in the drawings, and specific language will be used to describe these examples. It will nevertheless be understood that no limitation of the scope of the present disclosure is thereby intended. Any alteration and further modifications in the described embodiments, and any further applications of the principles of the invention as described herein, are contemplated as would normally occur to one skilled in the art to which the disclosure relates.

Presented herein are devices, systems, and methods that enable a healthcare provider to monitor the position and movement of anatomical structures within a patient's body. In one embodiment, the system comprises a sensor, a magnetic source, and a receiver. In some instances, the sensor is a device capable of detecting and measuring an electromagnetic field. The magnetic source is a source capable of producing an electromagnetic field, including both permanent magnets and electronic circuits. In one embodiment, the sensor is attached to an anatomical structure inside a patient's body while the magnetic source is located outside of the patient's body. In that regard, a magnitude of the magnetic field is measured by the sensor positioned within the body and communicated to the receiver. The measured changes in the magnitude of the magnetic field are utilized to track movement of the sensor and, thereby, the anatomical structure to which the sensor is attached. In some instances, the receiver is a device that is capable of communicating with the sensor. In some embodiments, the receiver is configured to communicate wirelessly with the sensor in order to receive information relating to magnitude or strength of the magnetic field being measured by the sensor. In some instances the receiver is located outside of the patient's body. In other embodiments the receiver is located inside of the patient's body as well. In one particular embodiment, the receiver is integrated with the sensor such that the sensor functions as both the sensor and the receiver. In other embodiments, the receiver is spaced from the sensor within the patient's body.

Alternatively, in another embodiment the magnetic source is attached to an anatomical structure inside a patient's body while the sensor is located outside of the patient's body. In that regard, a magnitude of the magnetic field generated by the magnetic source is measured by the sensor positioned outside the body. The measured changes in the magnitude of the magnetic field are utilized to track movement of the magnetic source and, thereby, the anatomical structure to which the magnetic source is attached. In some instances where the magnetic source is positioned within the patient's body, the sensor and the receiver are joined together to form a single external component. Further, in some instances the sensor and/or the receiver includes a processor programmed to calculate the relative movement and/or position of the magnetic source based on the detected changes in the magnitude of the magnetic field.

In operation, an implantable version of either the magnetic source or the sensor is attached to an anatomical structure while the other is positioned outside of the patient's body. In some instances, the magnetic source is configured to generate an electromagnetic field of known strength. The sensor detects and measures the strength of the electromagnetic field generated by the magnetic source. Therefore, changes in the absolute strength of the electromagnetic field as detected by the sensor are used to determine a position of the sensor relative to the magnetic source. In some instances, the system is calibrated in order to define an origin or zero point from which the movement of the anatomical feature is tracked. In that regard, in some instances the sensor is calibrated. Specifically, the sensor is calibrated such that a baseline strength for the electromagnetic field generated by the magnetic source is determined at certain distance between the sensor and the magnetic source. The sensor is then able to monitor changes in the strength of the electromagnetic field with respect to the baseline measurement. Relative changes in the strength of the measured electromagnetic field to the baseline measurement are utilized to determine the relative movement between the sensor and the magnetic source. In other instances, the magnetic source is calibrated. For example, in some instances the magnetic source is capable of generating magnetic fields of varying strength. Accordingly, with the sensor positioned at a known distance and/or orientation relative to the magnetic source the strength of the magnetic source is adjusted to a desired baseline level for the known distance and/or orientation. Subsequently, changes in the strength of the measured electromagnetic field to the baseline level are utilized to determine the relative movement between the sensor and the magnetic source. Thus, in this manner the position of the anatomical structure is monitored by measuring the changes in the magnetic field as measured by the sensor.

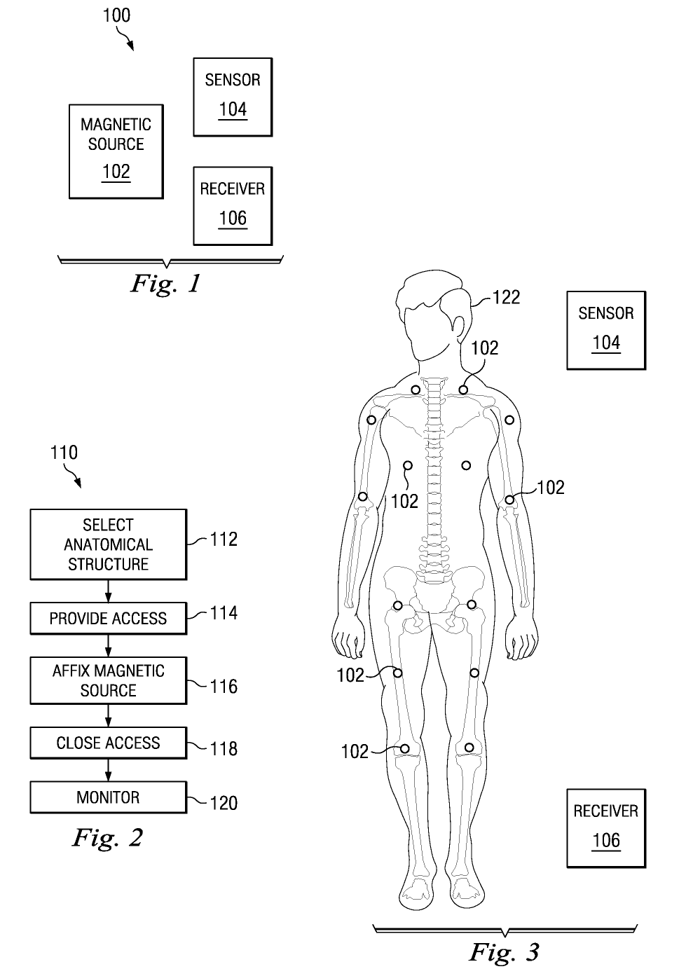

Referring to FIG. 1, shown therein is a schematic diagram of a system 100 for monitoring the position and movement of anatomical structures according to one embodiment of the present disclosure. In some instances, the system 100 enables a healthcare provider to monitor the position and movement of anatomical structures within a patient. In the illustrated embodiment, the system 100 includes an implantable magnetic source 102, a sensor 104, and a receiver 106. Additional aspects of the system 100 and its components will be apparent from the following.

In general, the magnetic source 102 is a device capable of producing a magnetic field. The magnetic field produced by magnetic source 102 generates magnetic field lines that extend outward from the magnetic source and are detectable by the sensor 104. The magnetic field propagated by magnetic source 102 is a type of electromagnetic radiation. While the present disclosure will focus its discussion on magnetic fields, it is understood that electromagnetic radiation includes other types of field lines, including without limitation radio waves, microwaves, infrared, visible light, ultraviolet, X-rays, and gamma rays. Accordingly, in other embodiments the concepts of the present disclosure may utilize types of electromagnetic radiation other than magnetic fields.

In some embodiments, the magnetic source 102 is a permanent magnet. The magnetic source 102 is selected from one or more of a neodymium iron boron magnet, a samarium cobalt magnet, an alnico magnet, and a ceramic or ferrite magnet in some instances. In that regard, the magnetic source 102 is a magnetic earth metal in some instances. In other embodiments, the magnetic source 102 is a temporary magnet. In some instances, the magnetic source 102 is an electromagnet. In that regard, the magnetic source 102 comprises an electrical circuit configured to generate a magnetic field in some instances. In that regard, the electrical circuit utilizes direct current in some embodiments and utilizes alternating current in other embodiments.

The implantable magnetic source 102 is sized and shaped to be affixed to one or more anatomical structures inside the patient's body. In use, the magnetic source 102 is affixed to anatomical structures within the patient's body that are to be monitored or tracked. These anatomical structures include bony anatomy including, but not limited to long bones, short bones, and any vertebra of the spinal column. The anatomical structures also include soft tissues, including muscles, tendons, ligaments, skin, heart, lungs, liver, bladder, kidneys, gall bladder, and the digestive tract including the esophagus, stomach, large intestine, and small intestine. With respect to the digestive track, in some instances the implantable magnetic source 102 is not affixed or attached to the anatomical structure, but instead is introduced into the digestive track and allowed to travel along the natural path created by the digestive track. As will be understood from the general description below regarding the tracking of movement of the magnetic sources of the present disclosure, the path of the magnetic source is tracked such that its relative position is utilized to establish a path associated with digestive track.

While, in many instances, the present disclosure will discuss the use of a single magnetic source 102, in some embodiments two or more magnetic sources are utilized. In that regard, in some embodiments at least three separate magnetic sources are utilized such that the positions of the magnetic sources is utilized to triangulate a relative position of a magnetic sensor or sensing system to the magnetic sources. In some instances, at least three separate magnetic sources are attached to different portions of an anatomical structure such that the movement of the anatomical structure is tracked by triangulating the positions of the magnetic sources. Further, in some instances a plurality of magnetic sources are utilized to form a magnetic field array.

In some instances, the magnetic source 102 is sized and shaped to be fixedly secured to anatomical structures within the body. In that regard, the magnetic source 102 includes features to facilitate engagement with an anatomical structure through the use of one or more of fasteners including, but not limited to screws, sutures, staples, and/or medical adhesives. Further, the magnetic source 102 is fixedly secured to an outer portion of the anatomical feature in some instances. In other instances, the magnetic source 102 is at least partially embedded within a portion of the anatomical structure. As one particular example, as discussed below with respect to FIGS. 9-14, in some instances magnetic source 102 is sized and shaped to threadingly engage and penetrate a bone thereby securely affixing the magnetic source to the bone.

Sensor 104 is a device capable of detecting and measuring a strength or magnitude of a magnetic field. In some instances, the sensor 104 includes one or more magnetometers. In that regard, the sensor is a Hall-effect sensor in some instances. In other instances, a fluxgate magnetometer is utilized. In some instances, the magnetometer is a magnetic sensor available from Yamaha, such as the YAS525B MS-1 magnetic field sensor, the YAS526C MS-2 magnetic field sensor, and/or the YAS529 MS-3C magnetic field sensor. In some instances, the magnetometer is a magnetic sensor available from Honeywell, such as the HMC1043 3-axis magnetic sensor, the HMC1501 magnetic sensor, the HMC1512 magnetic sensor, and/or the HMC5843 3-axis digital compass. In some instances, the magnetometer is a sensor available from Philips Semiconductor, such as the KMZ10A, the KMZ10A1, the KMZ10B, the KMZ10C, the KMZ51, and/or the KMZ52 magnetic sensors. In other instances, however, other magnetometers from other manufacturers are utilized.

While, in many instances, the present disclosure will discuss the use of a single sensor 104, in some embodiments two or more sensors are utilized. In that regard, in some embodiments a plurality of sensors are utilized to form a sensor array for monitoring the magnetic field. Further, the present disclosure utilizes the terms “sensor system” and “sensing system”. Unless otherwise stated, however, the terms “sensor system” and “sensing system” do not imply the use of multiple sensors. Rather, in some instances the terms “sensor system” or “sensing system” include only a single sensor.

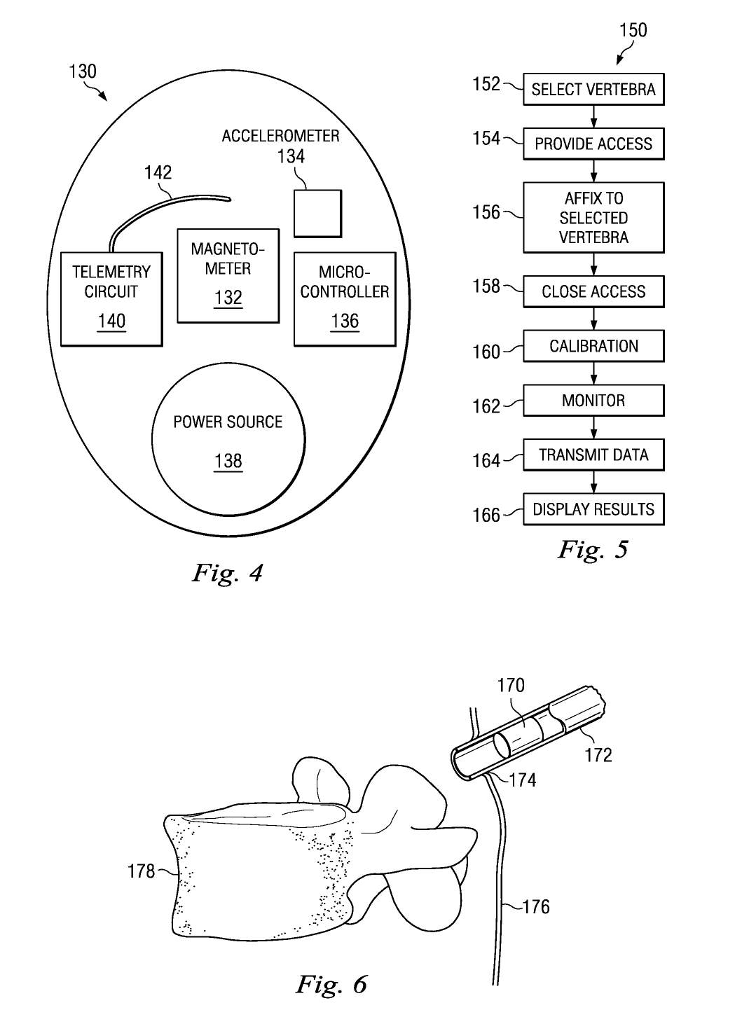

The sensor 104 is sized and shaped for positioning outside of the patient's body. In some instances, the sensor 104 is sized and shaped to be affixed to the skin surface of a patient in an area adjacent to the internal anatomical structure that has a magnetic source 102 affixed to it. One particular embodiment of such an external sensor is discussed in greater detail below with respect to FIG. 4. In some instances, the sensor 104 is sized and shaped to be affixed to the patient's clothing near the internal anatomical structure that has a magnetic source 102 affixed to it. Furthermore, in other embodiments sensor 104 is positioned outside of and spaced away from the patient's body. For example, FIG. 8 discussed below illustrates one such external sensor system that is spaced from the patient's body.

Finally, receiver 106 represents a device that is capable of communicating with sensor 104. In some instances, receiver 106 is capable of communicating with the sensor 104 via wireless telemetry. Generally, the receiver 106 is configured to communicate with sensor 104 in order to receive information relating to the strength of the magnetic field generated by the magnetic source 102 in order to track the position and movement of magnetic source and, thereby, track the position and movement of the anatomical feature to which the magnetic source is attached. In some instances, the receiver 106 is integrated with the sensor 104 into a single unit. In other instances, the receiver 106 is in wired communication with the sensor 104. In some embodiments, the receiver 106 includes software for calculating the relative movement of the magnetic source 102 relative to the sensor 104 based on the changes in the strength of the magnetic field as measured by the sensor 104. In that regard, the receiver 106 includes a processor and memory for storing and running the software in such embodiments. In some instances, the receiver 106 is a personal computer, handheld computer, or other computing device. In some instances, the receiver 106 includes or is connected to a display for displaying the position and movement information in human intelligible form.

In operation, magnetic source 102 is affixed to a patient's anatomical structure within the body and generates a magnetic field. Sensor 104 is positioned outside of the patient's body and measures the strength or magnitude of the magnetic field generated by magnetic source 102. The sensor 104 sends a representation of the measured strength to the receiver 106. In that regard, in some instances the strength of the magnetic field, as measured in gauss or otherwise, is converted into a corresponding electrical signal by the sensor. In such instances, the voltage of the electrical signal is proportional to the strength of the magnetic field. Accordingly, the receiver 106 detects the voltage output from the sensor 104 and calculates the relative movement of the magnetic source based on changes in the voltage output. In some instances, the receiver 106 interfaces with a computer program that monitors the strength of the magnetic field detected by sensor 104 based on the voltage output by the sensor in order to determine the position of the magnetic source 102 relative to the sensor 104. In other instances, the sensor 104 measures the strength of the magnetic field and converts it to a digital bit that is stored in memory accessible by the sensor 104. In some instances the memory is built into the sensor 104.

In some instances, the sensor 104 is calibrated with respect to the magnetic source 102. Specifically, the sensor 104 is calibrated such that a baseline strength for the magnetic field generated by magnetic source 102 is determined at a certain distance between the sensor 104 and magnetic source 102 and/or a known orientation of the patient relative to the sensor 104. Once the baseline strength is established, the position associated with the baseline measurement serves as an origin and the sensor 104 is able to monitor changes in the strength of the magnetic field with respect to the baseline measurement and determine movement relative to the origin. In that regard, the sensor 104 transmits the measured field data to receiver 106. Because sensor 104 is calibrated, the computer program can compare the sensor's measured strength of the magnetic field to the calibrated baseline strength of the magnetic field to determine the movement of the magnetic source 104 relative to origin defined by the baseline measurement. Accordingly, in this manner the relative changes of the measured magnetic field with respect to the calibrated baseline strength are used to track the movement of the anatomical structure to which magnetic source 102 is affixed. In other instances, the absolute value of the magnetic field is utilized to track movements of the magnetic source. In that regard, changes to the absolute value of the magnetic field as measured by the sensor 104 are utilized to track the movement of the magnetic source relative to the sensor 104.

Referring now to FIG. 2, shown therein is a flowchart of a method 110 for affixing a magnetic source to an anatomical structure and monitoring the position and movement of the anatomical structure according to one embodiment of the present disclosure. The method 110 begins at step 112 where an anatomical structure is selected to be monitored. The method 110 continues at step 114 where minimally invasive access to the selected anatomical structure is provided. In that regard, in some instances an incision is made through the patient's skin and a cannula is introduced through the incision and guided to a position adjacent to the selected anatomical structure. In other instances, access is achieved without the need for creating an incision. In that regard, access may be provided by entering a natural orifice of the patient in some instances. In general, any suitable method of accessing the selected anatomical structure is utilized, including non-minimally invasive approaches if necessary.

After gaining access to the selected anatomical structure, the method 110 continues at step 116 where the magnetic source 102 is affixed to the selected anatomical structure. As discussed above, magnetic source 102 can be affixed to the selected anatomical structure via screws, sutures, staples, and/or medical adhesives. In general, the manner in which the magnetic source is affixed to the selected anatomical structure is based on the type of anatomical structure selected and/or the treating medical personnel's preferences. In some instances, screws, staples, and/or medical adhesives are utilized to secure the magnetic source to bony anatomical structures. In some instances, sutures and/or staples are utilized to secure the magnetic source to soft tissue anatomical structures. After affixing the magnetic source 102 to the selected anatomical structure, the method continues at step 118 where the minimally invasive access to the anatomical structure is closed.

Finally, the method continues at step 120 where the sensor 104 is utilized to monitor the magnetic field generated by the implanted magnetic source 102 and, thereby, track the position of the selected anatomical structure to which the magnetic source has been affixed. Specifically, as discussed above, the sensor 104 detects and measures the strength of the magnetic field generated by magnetic source 102. The strength of the magnetic field detected by sensor 104 is then used to determine the relative position and movement of the magnetic source 102. Because magnetic source 102 is affixed to the selected anatomical structure, the relative or absolute strength of the electromagnetic field as detected by sensor 104 is utilized to determine the position and movement of the selected anatomical structure.

Referring now to FIG. 3, shown therein a front view of a patient 122 whose anatomical features are being monitored with a system for monitoring the position and movement of anatomical structures, incorporating aspects of the system 100 shown in FIG. 1 and the method 110 shown in FIG. 2. As shown, the sensor 104 and receiver 106 are positioned outside of the patient's body. In that regard, in some instances the sensor 104 is maintained in a fixed position. For example, in some instances the sensor 104 is mounted to a wall, positioned on a stand, or otherwise held in a stable position. Further, although shown as one sensor 104 positioned outside the body, in some instances the sensor 104 comprises a plurality of sensors. In that regard, in some instances the plurality of sensors operate independently. In other instances, the plurality of sensors operate in a coordinated manner as a sensor array. Further, while the sensor 104 is shown spaced from the patient 122, as discussed above, in other instances the sensor 104 is placed on the skin or clothing of the patient adjacent to the anatomical structure being monitored.

Additionally, FIG. 3 demonstrates that multiple magnetic sources 102 are affixed to a plurality of anatomical structures throughout the patient. Furthermore, it should be understood that in some instances one or more of the illustrated magnetic sources 102 comprises two or more magnetic sources affixed to a single anatomical structure. Where a plurality of magnetic sources are positioned within the patient as shown, in some instances only some of the magnetic sources are activated at any one time in order to prevent interference with tracking of the anatomical features. In other instances, the plurality of magnetic sources include different types of magnetic sources or identifiers such that the magnetic sources are distinguishable from one another such that the different anatomical features may be tracked by focusing on the magnetic sources of a particular type or having a particular identifier.

In active use, the magnetic source 102, located inside the patient's body, generates a magnetic field. The sensor 104, located outside of the patient's body, is excited by the magnetic field generated by magnetic source 102. Specifically, sensor 104 detects the magnetic field lines being produced by magnetic source 102. The sensor 104 transmits data representative of the magnitude of the magnetic field to receiver 106. As discussed above, the data is analog form (e.g., voltage) in some instances. In other instances, the data is in digital form (e.g., bits of data representative of the detected magnetic field). By monitoring the changes in the magnetic field as detected by the senor 104, the position of the magnetic source 102 affixed to the anatomical structure is tracked. Accordingly, in this manner the system 100 is utilized to detect the position and movement of anatomical structures within the patient 122.

Referring now to FIG. 4, shown therein is a schematic diagram of an external sensor 130 according to one embodiment of the present disclosure. In some instances, the sensor 130 is sized and shaped to be positioned outside to the patient's body. In some embodiments, the sensor 130 is sized and shaped to be affixed to the surface of the skin of a patient in area adjacent to a part of the patient's body containing an implanted magnetic source. In the illustrated embodiment, the sensor 130 includes a magnetometer 132, an accelerometer 134, a microcontroller 136, a power source 138, and a communication circuit 140. Magnetometer 132 measures the strength and/or direction of the electromagnetic field generated from the magnetic source 102 implanted in the patient's body. In that regard, the magnetometer is a Hall-effect sensor in some instances. In other instances, a fluxgate magnetometer is utilized. In some instances, the magnetometer is a magnetic sensor available from Yamaha, such as the YAS525B MS-1 magnetic field sensor, the YAS526C MS-2 magnetic field sensor, and/or the YAS529 MS-3C magnetic field sensor. In some instances, the magnetometer is a magnetic sensor available from Honeywell, such as the HMC1043 3-axis magnetic sensor, the HMC1501 magnetic sensor, the HMC1512 magnetic sensor, and/or the HMC5843 3-axis digital compass. In some instances, the magnetometer is a sensor available from Philips Semiconductor, such as the KMZ10A, the KMZ10A1, the KMZ10B, the KMZ10C, the KMZ51, and/or the KMZ52 magnetic sensors. In other instances, however, other magnetometers from other manufacturers are utilized.

The accelerometer 134 measures non-gravitational accelerations that are produced by mechanically accelerating the accelerometer. Accelerometer 134 can be a single or multi-axis model. Specifically, in some instances the accelerometer 134 is a three axis accelerometer that is able to detect the magnitude and direction of the acceleration as a vector quantity that can be used to sense the orientation and/or position of the sensor 130 as well as any vibrations imparted on the sensor.

The magnetometer 132 and accelerometer 134 are controlled by a microcontroller 136. Specifically, microcontroller 136 interfaces with the magnetometer 132 and accelerometer 134 to receive the data generated by the magnetometer and accelerometer. In some instances, the microcontroller 136 is a microcontroller available from Texas Instruments, such as the MSP430x20x1, the MSP430x20x2, and/or the MSP430x20x3 families of mixed signal microcontrollers. In other instances, however, other microcontrollers from other manufacturers are utilized. Furthermore, the microcontroller interfaces with a communication circuit 140 in order to communicate the data received from the magnetometer 132 and accelerometer 134 to an external device, such as receiver 106. In some instances, the sensor 130 includes memory for storing the data received from the magnetometer 132 and/or the accelerometer 134.

The communication circuit 140 is connected to an antenna 142 and is adapted for communicating with an external device, which will be referred to as receiver 106 for exemplary purposes. In particular, the communication circuit 140 is adapted for communicating wirelessly with a telemetry circuit of the receiver 106. In some instances, the wireless communication between the sensor 130 and the receiver 106 is accomplished using one or more of radio frequency identification (RFID), inductive telemetry, acoustic energy, near infrared energy, “Bluetooth,” computer network protocols, and other wireless communication protocols. In the present embodiment, the communication circuit 140 is adapted for RFID communication. In that regard, in some instances the telemetry circuit 140 is a passive RFID tag. Using a passive RFID tag limits the power requirements of the communication circuit 140 while still providing wireless communication of data from the sensor 130 to the receiver 106.

Supplying the power requirements of the sensor 130 is a power source 138. In the current embodiment, the power source 138 is a battery. The battery used as a power source 138 can be a lithium iodine battery similar to those used for other medical implant devices such as pacemakers. However, the battery power source 138 can be any type of battery. Further, the battery can be rechargeable. For example, the battery can be configured such that an applied electromagnetic field will recharge the battery. A rechargeable battery of this type extends the usable life of the sensor 130. It is also contemplated that the power source 138 can be a capacitor or an array of capacitors. Using a capacitor provides an alternative form of replenishable power to a rechargeable battery.

The power source 138 is connected to one or more of the magnetometer 132, accelerometer 134, microcontroller 136, and telemetry circuit 140. The power source 138 is connected to these components so as to allow monitoring by the sensor 130. In some instances, the power source 138 allows for continuous monitoring by the sensor 130. In other instances, the sensor 130 and/or the power source 138 are selectively activatable to monitor the magnetic field generated by the magnetic source 102. The magnetometer 132 and accelerometer 134 utilize the power source 138 to facilitate the sending of the measured data to the microcontroller 136 in some instances. The microcontroller 136, in turn, utilizes the power source 138 to process the data received from the magnetometer 132 and accelerometer 134 and to send the processed data to the telemetry circuit 140 for communication to the receiver 106.

In other embodiments the power source 138 can also be connected to the telemetry circuit 140 to provide power to facilitate communication with the receiver 106. However, in the present embodiment the telemetry circuit 140 does not require power from the power source 138 because it communicates with receiver 106 utilizing a passive RFID tag. Further, the power source 138 can be connected to other electronic components not found in the current embodiment. It is also fully contemplated that the power source 138 can include a plurality of batteries or other types of power sources. Finally, the sensor 130 is self-powered in some embodiments and does not require a separate power supply. For example, a piezoelectric transducer can be incorporated into sensor 130. In such an embodiment, the piezoelectric transducer detects the movement of the sensor 130 and converts it into an electrical signal sufficient to power the magnetometer 132, accelerometer 134, and/or microcontroller 136. Then, as in the current embodiment, the sensor 130 can utilize a passive RFID tag or other passive telemetry unit to communicate the data produced by the sensor to another device such as receiver 106. Thus, allowing the sensor 130 to function without a dedicated power source.

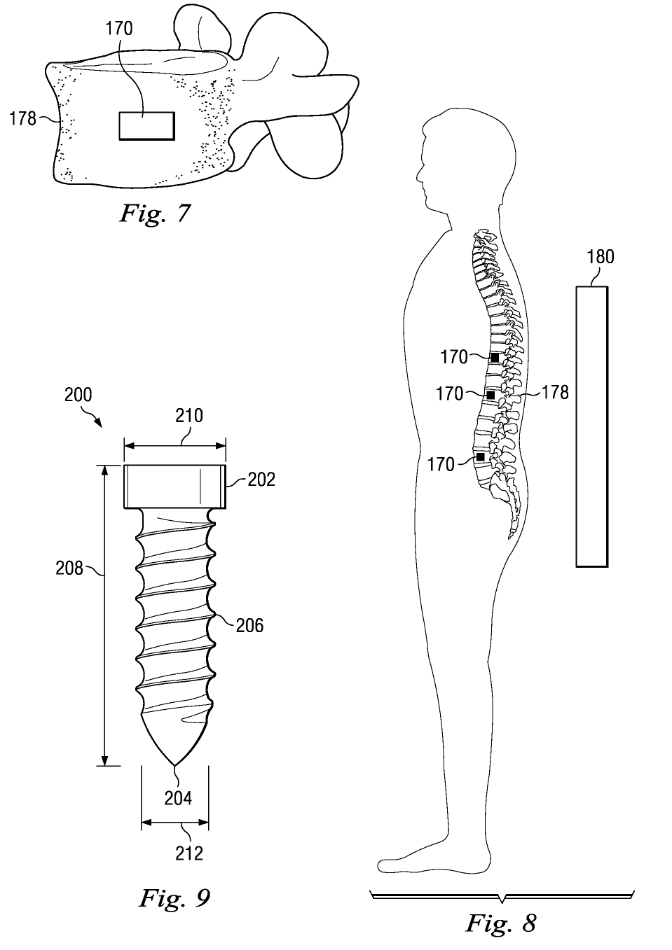

Referring now to FIGS. 5-8, shown therein is a method of affixing a magnetic source to vertebra of a spinal column to monitor the position and movement of the vertebra according to one embodiment of the present disclosure. In particular, FIG. 5 is a flowchart of a method 150 for affixing a magnetic source to a vertebra of a spinal column to monitor the position and movement of the vertebra; FIG. 6 is a partial cross-sectional, perspective view of a magnetic source being implanted adjacent a vertebra of a spinal column of a patient; FIG. 7 is a partial cross-sectional, front view similar that of FIG. 6, but showing the magnetic source engaged with the vertebra of the spinal column of the patient; and FIG. 8 is a side view of a system monitoring the position and movement of vertebrae of a patient after implantation of the magnetic source.

The method 150 begins at step 152 by selecting at least one vertebra of the spinal column for monitoring. For simplicity, the method 150 will be described with respect to a single vertebra. However, it is understood that similar steps are utilized to monitor two or more vertebrae, up to and including monitoring all of the vertebrae of the spinal column if desired. In some instances, the monitoring of the selected vertebra includes monitoring the position and relative flexion, extension, rotation, and/or right and left lateral bending of the vertebra within the spinal column. The method 150 continues at step 154 where minimally invasive access to selected vertebra is provided. For example, referring to FIG. 6, a magnetic source 170 is being introduced through a cannula 172 that extends through an incision 174 in the patient's skin 176 towards a vertebra 178. In some instances, a distal end of the cannula is engaged with the vertebra 178. The cannula provides a percutaneous access path for delivery of the magnetic source 170 to vertebra 178. In some instances, cannula 172 is used to position the magnetic source 170 adjacent to the vertebra 178 of a spinal column prior to affixing the magnetic source 170 to the vertebra. In that regard, the magnetic source 170 is sized and shaped to be inserted within and guided through cannula 172. In some instances, the magnetic source 170 has a generally cylindrical profile with a maximum diameter less than the inner diameter of the cannula 172. In some instances, the cannula 172 is non-metallic for ease of delivering the magnetic source 170 through the cannula.

Referring again to FIG. 5, the method 150 continues at step 156, where the magnetic source 170 is affixed to the selected vertebra 178. In general, the magnetic source 170 can be affixed to any portion of the selected vertebra. In some instances, the magnetic source 170 is affixed to the selected vertebra's spinous process, transverse process, lamina, pedicle, and/or vertebral body. Furthermore, magnetic source 170 is implanted within the cancellous bone of the vertebral body of the selected vertebra in some instances. As discussed above, affixing magnetic source 170 to the selected vertebra can be accomplished via screws, sutures, staples, and/or medical adhesives. Referring more particularly to FIG. 7, the magnetic source 170 is shown implanted within the cancellous bone of the vertebral body of vertebra 178. However, in other embodiments, magnetic source 170 can be inserted into and/or affixed to any portion of the vertebra including, but not limited to the spinous process, transverse process, lamina, pedicle, and/or vertebral body. In one particular embodiment, a magnetic source 170 is attached to each of the transverse processes and the spinous process of a vertebra. Magnetic source 170 can further be secured to vertebra 178 by injecting around the magnetic source medical adhesives.

Any number of medical adhesive can be injected around magnetic source 170 to secure the magnetic source to vertebra 178. For example, medical adhesives can include, but not limited to those made from polymethylmethacrylate (PMMA), calcium phosphate, hyrdroxyapatite-tricalcium phosphate (HA-TCP) compounds, bioactive glasses, polymerizable matrix comprising a bisphenol-A dimethacrylate, or CORTOSS™ by Orthovita of Malvern, Pa. (generically referred to as a thermoset cortical bone void filler). Calcium sulfate bone void fillers and other filling compositions or combinations of filling compositions can also be used. Bone void fillers or bone cements can be treated with biological additives such as demineralized bone matrix, collagen, gelatin, polysaccharide, hyaluronic acid, keratin, albumin, fibrin, cells and/or growth factors. Additionally or alternatively, bone void fillers or bone cements can be mixed with inorganic particles such as hydroxyapatite, fluorapatite, oxyapatite, wollastonite, anorthite, calcium fluoride, agrellite, devitrite, canasite, phlogopite, monetite, brushite, octocalcium phosphate, whitlockite, tetracalcium phosphate, cordierite, berlinite or mixtures thereof.

Other osteoinductive, osteoconductive, or carrier materials that can be injected, extruded, inserted, or deposited around magnetic source 170 and can include collagen, fibrin, albumin, karatin, silk, elastin, demineralized bone matrix, or particulate bone. Various bone growth promoting biologic materials can also be added to the any medical adhesive used to further secure magnetic source 170 to vertebra 178. For example, growth promoting biologic materials can include, but not limited to mysenchymal stem cells, hormones, growth factors such as transforming growth factor beta (TGFb) proteins, bone morphogenic proteins (including BMP and BMP2), or platelet derived growth factors.

Referring again to FIG. 5, after affixing the magnetic source 170 to the selected vertebra, the method 150 continues at step 158 where the minimally invasive access is closed. The method 150 then continues at step 160, where the system is calibrated. In some instances, the magnetic source 170 is calibrated in order to generate a magnetic field having desired magnitude relative to a sensor. For example, referring more particularly to FIG. 8, shown therein is a patient having magnetic sources 170 affixed to multiple vertebrae, including vertebra 178, standing in front of a sensing system 180 for monitoring the magnetic field(s) generated by the magnetic sources 170. In some instances, the strength of the magnetic field(s) generated by the magnetic sources 170 are adjustable such that with the patient a known distance from the sensing system 180, the strength of the magnetic field(s) are adjusted to a desired level as measured by the sensing system 180. In other instances, the sensing system 180 is calibrated with respect to the magnetic field generated by the magnetic sources 170. In that regard, the sensing system 180 is calibrated by determining a baseline strength of the electromagnetic field generated by magnetic sources 170 for a known position of the patient relative to the sensing system. In this manner, once the sensing system 180 is calibrated, it is able to detect changes in the strength of the magnetic field in order to monitor movement of the magnetic sources 170 relative to the sensing system.

Referring again to FIG. 5, the method 150 continues at step 162 where the sensing system 180 monitors the magnetic field being produced by magnetic source 170. By monitoring the strength of the magnetic field, sensing system 180 tracks the relative changes in the position and/or movement of the magnetic source 170 and the selected vertebra to which the magnetic source is attached. In some instances, the sensing system 180 stores the data generated from measurements of the magnetic field. In that regard, the sensing system 180 includes internal memory capable of storing data in some instances. The method 150 continues at step 164 where the sensing system 180 sends the data relating to the magnetic field to a receiver or processor. In some instances, the receiver or processor is part of the sensing system 180 such that the data is sent over wires or wirelessly within the sensing system 180. In other instances, the receiver or processor is separate from the sensing system 180. In some such instances, the data is sent wirelessly. As discussed above, the sensing systems of the present disclosure are configured with a transceiver to enable wireless communication to external devices in some instances. The sensing system is programmed to send the data automatically in some instances. In other instances, the sensing system sends the data as requested by the receiver, processor, or other external source.

Finally, the method 150 continues at step 166 where the results of the monitoring are displayed in human intelligible form. In some instances, the data is processed by a computer program in order to calculate 3-D images for the position and movement of the selected vertebra within the spinal column. Because the system is calibrated at step 160, the computer program can use the measurements of the strength of the magnetic field generated by the magnetic source 170 as measured by the sensing system 180 to determine the relative movement of the magnetic source over time. The movement of the magnetic source 170 is directly indicative of the movement of the selected vertebra since the magnetic source 170 is affixed to the vertebra. In this manner, the method 150 is utilized to track the position and movement of one or more vertebrae of a patient's spinal column.

Referring to FIGS. 9-14, shown therein are various views of a bone screw 200 having a magnetic source disposed therein according to one embodiment of the present disclosure. Referring more particularly to FIG. 9, shown therein is a side view of the bone screw 200. The bone screw 200 includes a head portion 202 defining a flange portion and an opposing leading end 204 that is sized and shaped for penetrating bone. To facilitate bone engagement the bone screw 200 includes threads 206 extending generally between the head portion 202 and the leading end 204. The threads 206 are configured to be of an appropriate size and shape to encourage bone engagement. As shown, the bone screw 200 has a length 208, which in some instances is between about 5.0 mm and about 50.0 mm. The head portion 202 of the bone screw 200 has a maximum width 210, which in some instances is between about 2.0 mm and about 25.0 mm. The threads 206 of the bone screw 200 have a maximum width 212, which in some instances is between about 2.0 mm and about 25.0 mm.

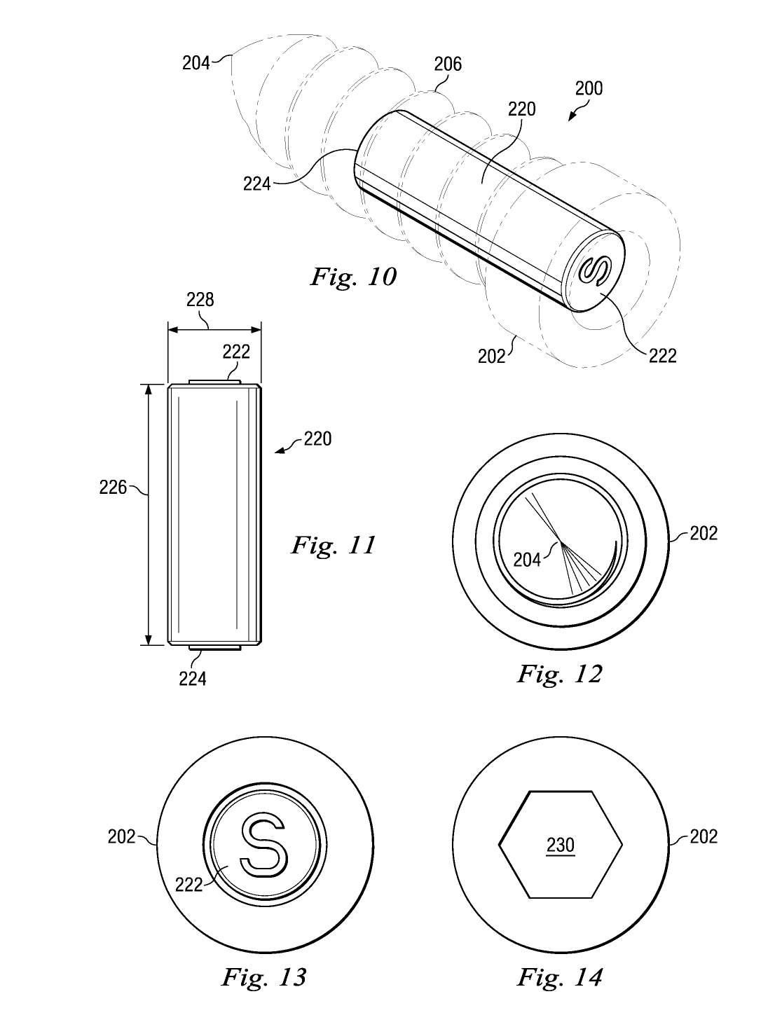

Referring to FIG. 10, shown therein is a perspective view of the bone screw of FIG. 9 with an outer housing of the bone screw 200 shown in phantom in order to highlight a magnetic source 220 disposed therein. The implantable bone screw 200 has an inner bore for receiving the magnetic source 220. As shown, the magnetic source 220 is positioned entirely within the inner bore of the bone screw 200. Magnetic source 220 is capable of producing a magnetic field having sufficient strength to be detectable outside of the patient's body. In the illustrated embodiment, the magnetic source 220 is a permanent magnet having a south pole 222 and an opposing north pole 224. Referring to FIG. 11, shown therein is a side view of the magnetic source 220 that is disposed within the bone screw 200. The magnetic source 220 has a length 226, which in some instances is between about 1.0 mm and about 49.0 mm. The magnetic source 220 also has a maximum width 228, which in some instances is between about 1.0 mm and about 24.0 mm. In other embodiments, the magnetic source and/or bone screw have lengths and widths outside of the disclosed ranges.

It is understood, however, that in other embodiments the magnetic source 220 is an electromagnet. For example, in some instances an active source is used to generate a magnetic field. Specifically, an active source, such as an electrical circuit, is utilized to generate the magnetic field in some instances. In that regard, the active source producing the magnetic field is able to adjust the strength of the electromagnetic field in some instances. In some embodiments, the strength is adjusted by varying the amount of current flowing through the electrical circuit. In such embodiments, the field can be shut off completely by stopping the flow of current through the circuit.

FIG. 12 is an end view of the leading end 204 of the bone screw 200, which, along with the threads 206, is the bone penetrating portion of the bone screw. FIG. 13 is an end view of the head portion 202 of the bone screw 200. In that regard, FIG. 13 illustrates a generic head portion 202 with no particular driver mating features. In general, it is understood that the head portion 202 includes features, such as recesses and/or projections, to facilitate driving of the bone screw 200 into bone. For example, referring to FIG. 14, shown therein is an example of a hex recess 230 for receiving a hex-driver for driving the bone screw 200 into bone. However, the head portion 202 may include any other suitable driving features in other embodiments.

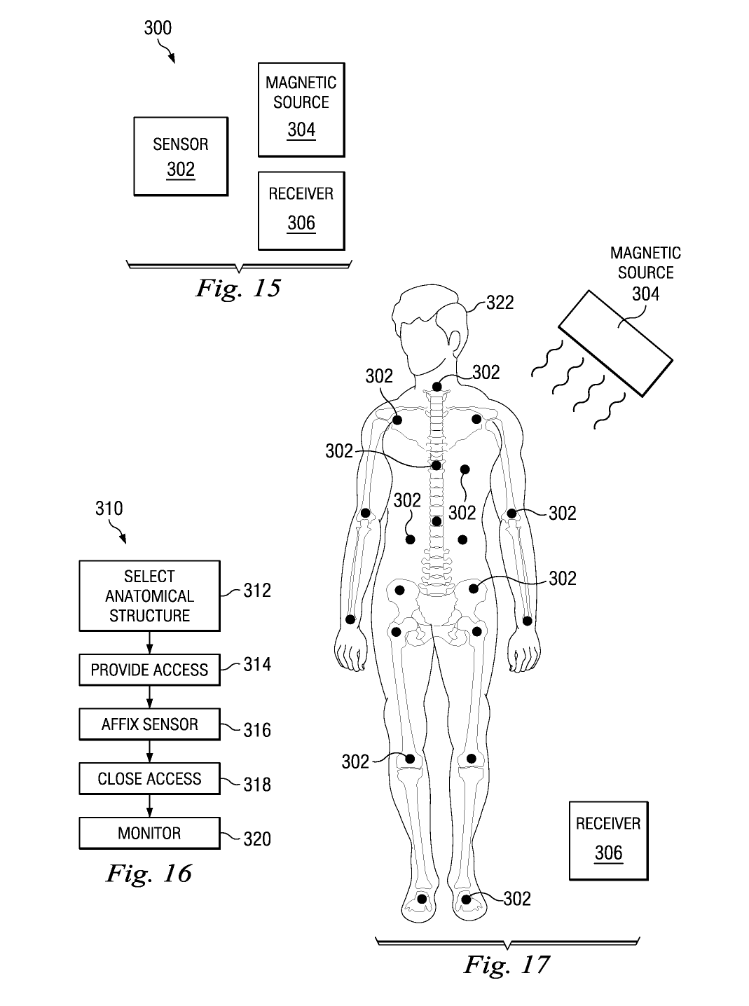

Referring now to FIG. 15, shown therein is a schematic diagram of a system 300 for monitoring the position and movement of anatomical structures according to another embodiment of the present disclosure. In some instances, the system 300 enables a healthcare provider to monitor the position and movement of anatomical structures within a patient. In the illustrated embodiment, system 300 includes an implantable sensor 302, a magnetic source 304, and a receiver 306. In many aspects, the functionality of the sensor 302, the magnetic source 304, and the receiver 306 are similar to the components discussed above with respect to system 100, the substantial differences between the systems 300 and 100 being that the sensor 302 is sized and shaped for implantation within a patient (whereas the sensor 104 was not necessarily sized and shaped for positioning inside the body) and that the magnetic source 304 does not need to be sized or shaped for implantation with the patient, but rather can be a relative large magnetic source (whereas the magnetic source 102 was sized and shaped for positioning inside the body). Accordingly, as described below, the system 300 operates in a manner substantially opposite that of system 100 such that the magnetic field is generated by the magnetic source 304 outside the body and detected by the sensor 302 affixed to an anatomical structure inside the body.

Sensor 302 is a device capable of detecting and measuring a strength or magnitude of a magnetic field. In some instances, the sensor 302 includes one or more magnetometers. In that regard, the sensor 302 is a Hall-effect sensor in some instances. In other instances, a fluxgate magnetometer is utilized. In some instances, the magnetometer is a magnetic sensor available from Yamaha, such as the YAS525B MS-1 magnetic field sensor, the YAS526C MS-2 magnetic field sensor, and/or the YAS529 MS-3C magnetic field sensor. In some instances, the magnetometer is a magnetic sensor available from Honeywell, such as the HMC1043 3-axis magnetic sensor, the HMC1501 magnetic sensor, the HMC1512 magnetic sensor, and/or the HMC5843 3-axis digital compass. In some instances, the magnetometer is a sensor available from Philips Semiconductor, such as the KMZ10A, the KMZ10A1, the KMZ10B, the KMZ10C, the KMZ51, and/or the KMZ52 magnetic sensors. In other instances, however, other magnetometers from other manufacturers are utilized.

While, in many instances, the present disclosure will discuss the use of a single sensor 302, in some embodiments two or more sensors are utilized. In that regard, in some embodiments a plurality of sensors are utilized to form a sensor array for monitoring the magnetic field. In that regard, in some embodiments at least three separate sensors are utilized such that the positions of the sensors are utilized to triangulate a relative position of a magnetic source to the sensors. In some instances, at least three separate sensors are attached to different portions of an anatomical structure such that the movement of the anatomical structure is tracked by triangulating the positions of the sensors. Further, in some instances a plurality of sensors are utilized to form a sensor field array.

The implantable sensor 302 is sized and shaped to be affixed to one or more anatomical structures inside the patient's body. In use, the sensor 302 is affixed to anatomical structures within the patient's body that are to be monitored or tracked. These anatomical structures include bony anatomy including, but not limited to long bones, short bones, and any vertebra of the spinal column. The anatomical structures also include soft tissues, including muscles, tendons, ligaments, skin, heart, lungs, liver, bladder, kidneys, gall bladder, and the digestive tract including the esophagus, stomach, large intestine, and small intestine. With respect to the digestive track, in some instances the implantable sensor 302 is not affixed or attached to the anatomical structure, but instead is introduced into the digestive track and allowed to travel along the natural path created by the digestive track. As will be understood from the general description below regarding the tracking of movement of the magnetic sources of the present disclosure, the path of the sensor is tracked such that its relative position is utilized to establish a path associated with digestive track.

In some instances, the sensor 302 is sized and shaped to be fixedly secured to anatomical structures within the body. In that regard, the sensor 302 includes features to facilitate engagement with an anatomical structure through the use of one or more of fasteners including, but not limited to screws, sutures, staples, and/or medical adhesives. Further, the sensor 302 is fixedly secured to an outer portion of the anatomical feature in some instances. In other instances, the sensor 302 is at least partially embedded within a portion of the anatomical structure. As one particular example, as discussed below with respect to FIGS. 22-25, in some instances the sensor 302 is sized and shaped to threadingly engage and penetrate a bone thereby securely affixing the sensor to the bone.

In general, the magnetic source 304 is a device capable of producing a magnetic field. The magnetic field produced by magnetic source 304 generates magnetic field lines that extend outward from the magnetic source and are detectable by the sensor 302. The magnetic field propagated by magnetic source 304 is a type of electromagnetic radiation. While the present disclosure will focus its discussion on magnetic fields, it is understood that electromagnetic radiation includes other types of field lines, including without limitation radio waves, microwaves, infrared, visible light, ultraviolet, X-rays, and gamma rays. Accordingly, in other embodiments the concepts of the present disclosure may utilize types of electromagnetic radiation other than magnetic fields.

In some embodiments, the magnetic source 304 is a permanent magnet or an array of permanent magnets. The magnetic source 304 is selected from one or more of a neodymium iron boron magnet, a samarium cobalt magnet, an alnico magnet, and a ceramic or ferrite magnet in some instances. In that regard, the magnetic source 304 is a magnetic earth metal in some instances. In other embodiments, the magnetic source 304 is a temporary magnet. In some instances, the magnetic source 304 is an electromagnet or an electromagnetic field generator. In that regard, the magnetic source 304 comprises an electrical circuit configured to generate a magnetic field in some instances. In that regard, the electrical circuit utilizes direct current in some embodiments and utilizes alternating current in other embodiments. Further, in some instances the magnetic source 304 is the earth's magnetic field.

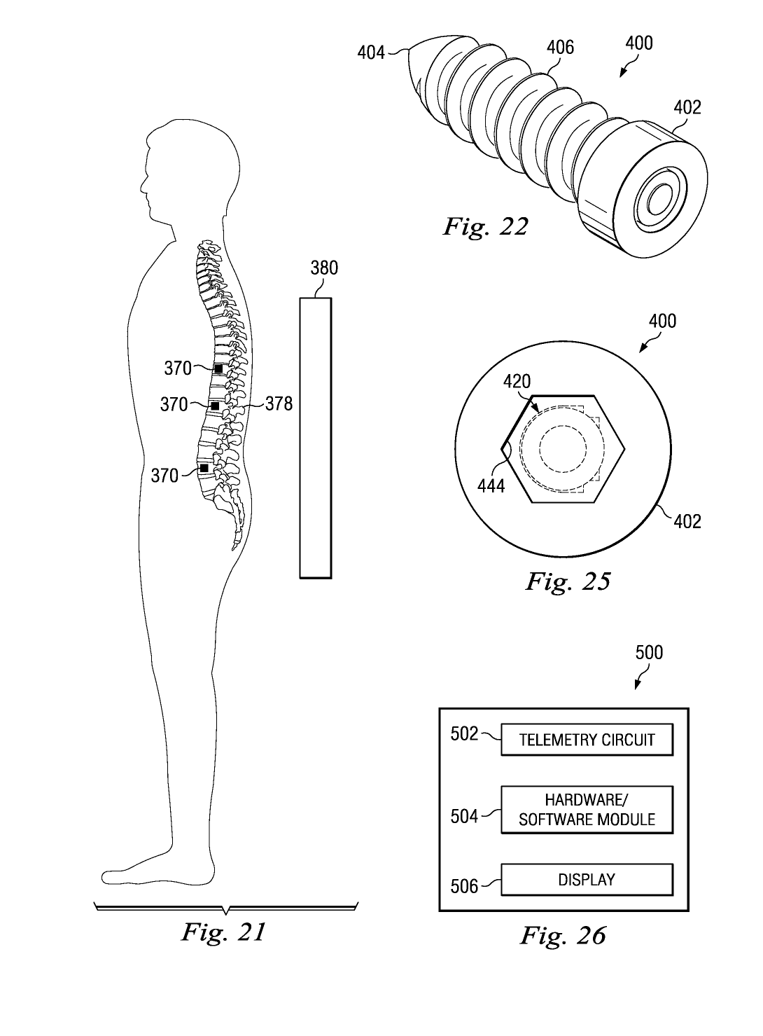

The magnetic source 304 is sized and shaped for positioning outside of the patient's body. In some embodiments magnetic source 304 is positioned outside of and spaced away from the patient's body, as shown in FIGS. 17 and 21. In other instances, the magnetic source 304 is sized and shaped to be affixed to the skin surface of a patient in an area adjacent to the internal anatomical structure that has a sensor 302 affixed to it. In some instances, the magnetic source 304 is sized and shaped to be affixed to the patient's clothing near the internal anatomical structure that has a magnetic source 102 affixed to it.

Finally, receiver 306 represents a device that is capable of communicating with sensor 302. In some instances, receiver 306 is capable of communicating with the sensor 302 via wireless telemetry. Generally, the receiver 306 is configured to communicate with sensor 302 in order to receive information relating to the strength of the magnetic field generated by the magnetic source 304 in order to track the position and movement of sensor relative to the magnetic source 304 and, thereby, track the position and movement of the anatomical feature to which the sensor 302 is attached. In some instances, the receiver 306 is integrated with the sensor 302 into a single unit. In other instances, the receiver 306 is in wired communication with the sensor 302 within the body of the patient. In some embodiments, the receiver 306 includes software for calculating the relative movement of the sensor 302 relative to the magnetic source 304 based on the changes in the strength of the magnetic field as measured by the sensor 302. In that regard, the receiver 306 includes a processor and memory for storing and running the software in such embodiments. In some instances, the receiver 306 is a personal computer, handheld computer, or other computing device. In some instances, the receiver 306 includes or is connected to a display for displaying the position and movement information in human intelligible form.

In operation, magnetic source 304 located outside of the patient's body generates a magnetic field directed at the patient. Sensor 302 is affixed to a patient's anatomical structure and measures the strength of the electromagnetic field generated by magnetic source 304. The sensor 304 either stores data representing the measured strength of the magnetic field or sends a representation of the measured strength to the receiver 306. In that regard, in some instances the strength of the magnetic field, as measured in gauss or otherwise, is converted into a corresponding electrical signal by the sensor. In such instances, the voltage of the electrical signal is proportional to the strength of the magnetic field. Accordingly, the receiver 306 detects the voltage output from the sensor 302 and calculates the relative movement of the magnetic source based on changes in the voltage output. In some instances, the receiver 306 interfaces with a computer program that monitors the strength of the magnetic field detected by sensor 302 based on the voltage output by the sensor in order to determine the position of the sensor 302 relative to the magnetic source 304. In other instances, the sensor 304 measures the strength of the magnetic field and converts it to a digital bit that is stored in memory accessible by the sensor 104. In some instances the memory is built into the sensor 104.

In some instances, the sensor 302 is calibrated with respect to the magnetic source 304. Specifically, the sensor 302 is calibrated such that a baseline strength for the magnetic field generated by magnetic source 304 is determined at a certain distance between the sensor 302 and magnetic source 304 and/or a known orientation of the patient relative to the magnetic source 304. Once the baseline strength is established, the position associated with the baseline measurement serves as an origin and the sensor 302 is able to monitor changes in the strength of the magnetic field with respect to the baseline measurement and determine movement relative to the origin. In that regard, the sensor 302 transmits the measured field data to receiver 306 in some instances. Because sensor 302 is calibrated, the receiver 306 can compare the measured strength of the magnetic field to the calibrated baseline strength of the magnetic field to determine the movement of the sensor 302 relative to origin defined by the baseline measurement. Accordingly, in this manner the relative changes of the measured magnetic field with respect to the calibrated baseline strength are used to track the movement of the anatomical structure to which sensor 302 is affixed. In other instances, the absolute value of the magnetic field is utilized to track movements of the magnetic source. In that regard, changes to the absolute value of the magnetic field as measured by the sensor 302 are utilized to track the movement of the sensor relative to the magnetic source 304. In some instances, the magnetic source 304 is the earth's magnetic field and the sensor 302 is a magnetic compass. In some instances, the sensor 302 is configured to determine its position based on the measured strength and polarity of the earth's magnetic field.

Referring now to FIG. 16, shown therein is a flowchart of a method 310 for affixing a sensor to an anatomical structure and monitoring the position and movement of the anatomical structure according to one embodiment of the present disclosure. The method 310 begins at step 312 where an anatomical structure is selected to be monitored. The method 310 continues at step 314 where minimally invasive access to the selected anatomical structure is provided. In that regard, in some instances an incision is made through the patient's skin and a cannula is introduced through the incision and guided to a position adjacent to the selected anatomical structure. In other instances, access is achieved without the need for creating an incision. In that regard, access may be provided by entering a natural orifice of the patient in some instances. In general, any suitable method of accessing the selected anatomical structure is utilized, including non-minimally invasive approaches if necessary.

After gaining access to the selected anatomical structure, the method 310 continues at step 316 where the sensor 302 is affixed to the selected anatomical structure. As discussed above, sensor 302 can be affixed to the selected anatomical structure via screws, sutures, staples, and/or medical adhesives. In general, the manner in which the sensor is affixed to the selected anatomical structure is based on the type of anatomical structure selected and/or the treating medical personnel's preferences. In some instances, screws, staples, and/or medical adhesives are utilized to secure the sensor to bony anatomical structures. In some instances, sutures and/or staples are utilized to secure the sensor to soft tissue anatomical structures. After affixing the sensor 302 to the selected anatomical structure, the method 310 continues at step 318 where the minimally invasive access to the anatomical structure is closed.

Finally, the method 310 continues at step 320 where the sensor 302 is utilized to monitor the magnetic field generated by a magnetic source 304 and, thereby, track the position of the selected anatomical structure to which the sensor has been affixed. Specifically, as discussed above, the sensor 302 detects and measures the strength of the magnetic field generated by magnetic source 304. The strength of the magnetic field detected by sensor 302 is then used to determine the relative position and movement of the sensor 302 relative to the magnetic source 304. Because sensor 302 is affixed to the selected anatomical structure, the relative or absolute strength of the magnetic field as detected by sensor 302 is utilized to determine the position and movement of the selected anatomical structure.

Referring now to FIG. 17, shown therein a front view of a patient 322 whose anatomical features are being monitored with a system for monitoring the position and movement of anatomical structures, incorporating aspects of the system 300 shown in FIG. 15 and the method 310 shown in FIG. 16. As shown, the magnetic source 304 and receiver 306 are positioned outside of the patient's body. In that regard, in some instances the magnetic source 304 is maintained in a fixed position. For example, in some instances the magnetic source 304 is mounted to a wall, positioned on a stand, or otherwise held in a stable position. Further, although shown as one magnetic source 304 positioned outside the body, in some instances the magnetic source 304 comprises a plurality of magnetic sources. In that regard, in some instances the plurality of magnetic sources operate independently. In other instances, the plurality of magnetic sources operate in a coordinated manner as a magnetic source array. Further, while the magnetic source 304 is shown spaced from the patient 322, as discussed above, in other instances the magnetic source 304 is placed on the skin or clothing of the patient adjacent to the anatomical structure being monitored.

Additionally, FIG. 17 demonstrates that multiple implantable sensors 302 are affixed to a plurality of anatomical structures throughout the patient. Furthermore, it should be understood that in some instances one or more of the illustrated sensors 302 comprises two or more magnetic sources affixed to a single anatomical structure. Where a plurality of sensors are positioned within the patient as shown, in some instances only some of the sensors are activated at any one time in order to prevent interference with tracking of the anatomical features. In other instances, the plurality of sensors include different types of unique identifiers such that the sensors are distinguishable from one another such that the different anatomical features may be tracked by focusing on the sensors having a particular identifier. Further, in some instances a sensor, such as that described with respect to FIG. 4, is positioned on a skin surface of the patient. The position of the sensor positioned on the skin of the patient is tracked in a similar manner and utilized to approximate the position of an adjacent internal anatomical feature with a completely non-invasive procedure.

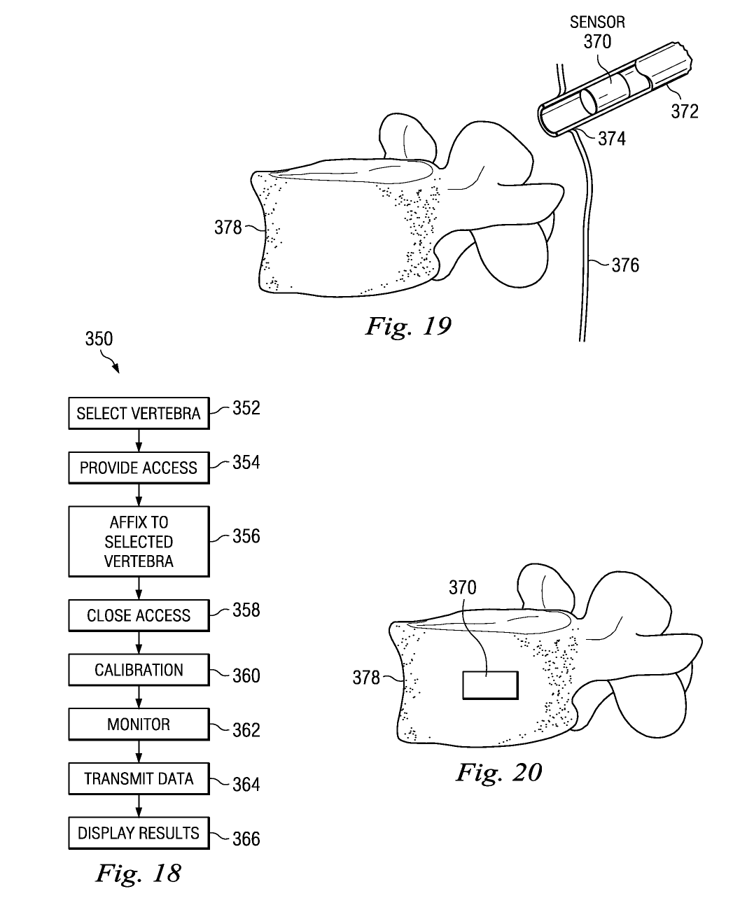

Referring now to FIGS. 18-21, shown therein is a method of affixing a sensor to vertebra of a spinal column to monitor the position and movement of the vertebra according to one embodiment of the present disclosure. In particular, FIG. 18 is a flowchart of a method 350 for affixing a sensor to a vertebra of a spinal column to monitor the position and movement of the vertebra; FIG. 19 is a partial cross-sectional, perspective view of a sensor being implanted adjacent a vertebra of a spinal column of a patient; FIG. 20 is a partial cross-sectional, front view similar that of FIG. 19, but showing the sensor engaged with the vertebra of the spinal column of the patient; and FIG. 20 is a side view of a system monitoring the position and movement of vertebrae of a patient after implantation of the sensor.

Referring to FIG. 18, the method 350 begins at step 352 by selecting at least one vertebra of the spinal column for monitoring. For simplicity, the method 350 will be described with respect to a single vertebra. However, it is understood that similar steps are utilized to monitor two or more vertebrae, up to and including monitoring all of the vertebrae of the spinal column if desired. In some instances, the monitoring of the selected vertebra includes monitoring the position and relative flexion, extension, rotation, and/or right and left lateral bending of the vertebra within the spinal column. The method 350 continues at step 354 where minimally invasive access to selected vertebra is provided. For example, referring to FIG. 19, a sensor 370 is being introduced through a cannula 372 that extends through an incision 374 in the patient's skin 376 towards a vertebra 378. In some instances, a distal end of the cannula is engaged with the vertebra 378. The cannula provides a percutaneous access path for delivery of the sensor 370 to vertebra 378. In some instances, cannula 372 is used to position the sensor 370 adjacent to the vertebra 378 of a spinal column prior to affixing the sensor 370 to the vertebra. In that regard, the sensor 370 is sized and shaped to be inserted within and guided through cannula 372. In some instances, the sensor 370 has a generally cylindrical profile with a maximum diameter less than the inner diameter of the cannula 372.

Referring again to FIG. 18, the method 350 continues at step 356, where the sensor 370 is affixed to the selected vertebra 378. In general, the sensor 370 can be affixed to any portion of the selected vertebra. In some instances, the sensor 370 is affixed to the selected vertebra's spinous process, transverse process, lamina, pedicle, and/or vertebral body. Furthermore, sensor 370 is implanted within the cancellous bone of the vertebral body of the selected vertebra in some instances. As discussed above, affixing sensor 370 to the selected vertebra can be accomplished via screws, sutures, staples, and/or medical adhesives. Referring more particularly to FIG. 20, the sensor 370 is shown implanted within the cancellous bone of the vertebral body of vertebra 378. However, in other embodiments, sensor 370 can be inserted into and/or affixed to any portion of the vertebra including, but not limited to the spinous process, transverse process, lamina, pedicle, and/or vertebral body. In one particular embodiment, a sensor 370 is attached to each of the transverse processes and the spinous process of a vertebra. Sensor 170 can further be secured to vertebra 378 by injecting around the magnetic source medical adhesives.

Any number of medical adhesive can be injected around sensor 370 to secure the sensor to vertebra 378. For example, medical adhesives can include, but not limited to those made from polymethylmethacrylate (PMMA), calcium phosphate, hyrdroxyapatite-tricalcium phosphate (HA-TCP) compounds, bioactive glasses, polymerizable matrix comprising a bisphenol-A dimethacrylate, or CORTOSS™ by Orthovita of Malvern, Pa. (generically referred to as a thermoset cortical bone void filler). Calcium sulfate bone void fillers and other filling compositions or combinations of filling compositions can also be used. Bone void fillers or bone cements can be treated with biological additives such as demineralized bone matrix, collagen, gelatin, polysaccharide, hyaluronic acid, keratin, albumin, fibrin, cells and/or growth factors. Additionally or alternatively, bone void fillers or bone cements can be mixed with inorganic particles such as hydroxyapatite, fluorapatite, oxyapatite, wollastonite, anorthite, calcium fluoride, agrellite, devitrite, canasite, phlogopite, monetite, brushite, octocalcium phosphate, whitlockite, tetracalcium phosphate, cordierite, berlinite or mixtures thereof.

Other osteoinductive, osteoconductive, or carrier materials that can be injected, extruded, inserted, or deposited around sensor 370 and can include collagen, fibrin, albumin, karatin, silk, elastin, demineralized bone matrix, or particulate bone. Various bone growth promoting biologic materials can also be added to the any medical adhesive used to further secure sensor 370 to vertebra 178. For example, growth promoting biologic materials can include, but not limited to mysenchymal stem cells, hormones, growth factors such as transforming growth factor beta (TGFb) proteins, bone morphogenic proteins (including BMP and BMP2), or platelet derived growth factors.

Referring again to FIG. 18, after affixing the sensor 370 to the selected vertebra, the method 350 continues at step 358 where the minimally invasive access is closed. The method 350 then continues at step 360, where the system is calibrated. In some instances, a magnetic source of the system is calibrated in order to generate a magnetic field having desired magnitude relative to the sensor 370. For example, referring more particularly to FIG. 21, shown therein is a patient having sensors 370 affixed to multiple vertebrae, including vertebra 378, standing in front of a magnetic source 380 for generating a magnetic field to be detected by the sensors 370. In some instances, the strength of the magnetic field generated by the magnetic source 380 is adjustable such that with the patient a known distance from the magnetic source 180, the strength of the magnetic field is adjusted to a desired level as measured by one or more of the sensors 370. In other instances, the sensors 370 are calibrated with respect to the magnetic field generated by the magnetic source 380. In that regard, the sensors 370 are calibrated by determining a baseline strength of the electromagnetic field generated by magnetic source 380 for a known position of the patient relative to the magnetic source. In this manner, once the sensors 370 are calibrated, the sensors are able to detect changes in the strength of the magnetic field in order to monitor movement of the sensors 370 relative to the fixed magnetic source 380.

Referring again to FIG. 18, the method 350 continues at step 362 where the sensors 370 monitor the magnetic field being produced by magnetic source 380. By monitoring the strength of the magnetic field, sensors 370 track the relative changes in the position and/or movement of the sensors 370 and the selected vertebra to which the sensors are attached. In some instances, the sensors 370 store the data generated from measurements of the magnetic field. In that regard, the sensors 370 include internal memory capable of storing data in some instances. In other instances, each of the sensors 370 is in communication with a single memory device for storing the data from all of the sensors. The method 350 continues at step 364 where the sensors 370 send the data relating to the magnetic field to a receiver or processor. In some instances, the receiver or processor is part of the sensors 370 such that the data is sent over wires or wirelessly within the sensors 370. In other instances, the receiver or processor is separate from the sensors 370. In some such instances, the data is sent wirelessly. As discussed above, the sensors of the present disclosure are configured with a transceiver to enable wireless communication to external devices in some instances. The sensors are programmed to send the data automatically in some instances. In other instances, the sensing system sends the data as requested by the receiver, processor, or other external source.

Finally, the method 350 continues at step 366 where the results of the monitoring are displayed in human intelligible form. In some instances, the data is processed by a computer program in order to calculate 3-D images for the position and movement of the selected vertebra within the spinal column. Because the system is calibrated at step 360, the computer program can use the measurements of the strength of the magnetic field generated by the magnetic source 380 as measured by the sensors 370 to determine the relative movement of the sensors 370 over time. The movement of the sensors 370 is directly indicative of the movement of the selected vertebrae since the sensors 370 are affixed to the vertebra. In this manner, the method 350 is utilized to track the position and movement of one or more vertebrae of a patient's spinal column.

Referring to FIGS. 22-25, shown therein are various views of a bone screw 400 having a sensor disposed therein according to one embodiment of the present disclosure. Referring more particularly to FIG. 22, shown therein is a perspective view of the bone screw 400. The bone screw 400 includes a head portion 402 defining a flange portion and an opposing leading end 404 that is sized and shaped for penetrating bone. To facilitate bone engagement the bone screw 400 includes threads 406 extending generally between the head portion 402 and the leading end 404. The threads 406 are configured to be of an appropriate size and shape to encourage bone engagement. As shown in FIG. 24, the bone screw 400 has a length 408, which in some instances is between about 5.0 mm and about 50.0 mm. The head portion 402 of the bone screw 400 has a maximum width 410, which in some instances is between about 2.0 mm and about 25.0 mm. The threads 406 of the bone screw 400 have a maximum width 412, which in some instances is between about 2.0 mm and about 25.0 mm.

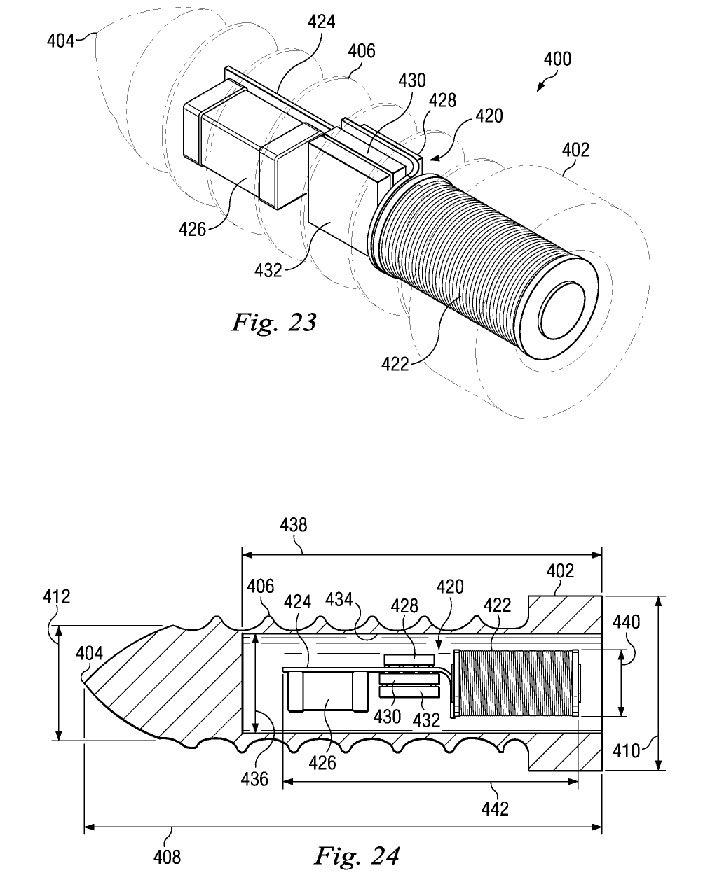

Referring more particularly to FIG. 23, shown therein is a perspective view of the bone screw 400 with an outer housing of the bone screw 400 shown in phantom in order to highlight a sensor 420 disposed therein. The implantable bone screw 400 has an inner bore for receiving the sensor 420. As shown, the sensor 420 is positioned entirely within the inner bore of the bone screw 400. Sensor 420 is capable of detecting and measuring the strength or magnitude of a magnetic field. In the illustrated embodiment, the sensor 420 includes a communication coil 422 that is attached to a plate 424. In general, the plate 424 serves to provide support for and interconnect the components of the sensor 420. In some instances the communication coil 422 is configured to both communicate wirelessly with a remote device, such as receivers 106 and 306, as well as wirelessly receive power for the sensor 420. In that regard, in some instances the communication coil 422 also serves as an energy harvesting coil. In some embodiments, the communication coil 422 harvests energy for the sensor 420 through inductive coupling with an external device. In some instances, the communication coil harvest energy from radio frequency communications. In some instances, the energy generated by the magnetic field that is being monitored by the sensor 420 is harvested by the communication coil in order to power the sensor. This allows the sensor 420 to remain in a dormant state for most of the time and power up when activated via an energy transfer. Once data transfer and/or tracking is completed the sensor 420 is able to return to its dormant state. In that regard, the sensor 420 also includes an energy storage device 426 that is in electrical communication with the communication coil 422. The energy storage device 426 is configured to store the energy harvested by the communication coil 422 for use by the sensor 420. In some instances, the energy storage device 426 is a capacitor. In some instances, the energy storage device 426 is a rechargeable battery. The energy storage device 426 is connected to one or more of the magnetometer 428, coil member 422, microcontroller 432 and component array network 430 in some instances.

The sensor 420 also includes a magnetometer 428 for detecting the presence and strength of the magnetic field. In that regard, the magnetometer 428 is a Hall-effect sensor in some instances. In other instances, a fluxgate magnetometer is utilized. In some instances, the magnetometer is a magnetic sensor available from Yamaha, such as the YAS525B MS-1 magnetic field sensor, the YAS526C MS-2 magnetic field sensor, and/or the YAS529 MS-3C magnetic field sensor. In some instances, the magnetometer is a magnetic sensor available from Honeywell, such as the HMC1043 3-axis magnetic sensor, the HMC1501 magnetic sensor, the HMC1512 magnetic sensor, and/or the HMC5843 3-axis digital compass. In some instances, the magnetometer is a sensor available from Philips Semiconductor, such as the KMZ10A, the KMZ10A1, the KMZ10B, the KMZ10C, the KMZ51, and/or the KMZ52 magnetic sensors. In other instances, however, other magnetometers from other manufacturers are utilized.

The sensor 420 also includes a component array network 430. The component array network 430 includes a plurality of passive components, such as resistors and capacitors, into a single device. The component array network 430 is utilized to condition and/or filter the electrical signals being communicated between the components of the sensor 420 in some instances. The sensor 420 also includes a processor or microcontroller 432. The microcontroller 432 interfaces with the magnetometer 428 to receive the data generated by the magnetometer 428 in response to the measured strength of the magnetic field. In some instances, the microcontroller 432 interfaces with communication coil 422 in order to facilitate transmission of the data to an external receiver. In some instances, the microcontroller 136 is a microcontroller available from Texas Instruments, such as the MSP430x20x1, the MSP430x20x2, and/or the MSP430x20x3 families of mixed signal microcontrollers. In other instances, however, other microcontrollers from other manufacturers are utilized.

As shown in FIG. 24, the sensor 420 is disposed within the inner bore of the bone screw 400. In that regard, the inner bore of the bone screw 400 has a width 436 and a length 438. The sensor 420, in turn, has a width 440 that is substantially equal to or less than the width 436 or the inner bore. In some instances, the width 440 of the sensor is between about 1.0 mm and about 24.0 mm. Similarly, the sensor 420 has a length 442 that is substantially equal to or less than the length 438 of the inner bore. In some instances, the length 442 of the sensor is between about 1.0 mm and about 49.0 mm. In other embodiments, the sensor and/or bone screw have lengths and widths outside of the disclosed ranges. Referring to FIG. 25, shown therein is an end view of the head portion 402 of the bone screw 400 according to one embodiment of the present disclosure. In that regard, the end portion 402 includes a hex recess 444 for receiving a hex-driver for driving the bone screw 400 into bone. However, it is understood the head portion 402 may include any other suitable driving features, as would be apparent to one skilled in the art, in other embodiments.

FIG. 26 is a schematic diagram of a receiver according to one embodiment of the present disclosure. Receiver 500 is capable of communicating with a sensor of the present disclosure. In some instances, receivers 106 and 306 discussed above include features similar to those described herein with respect to receiver 500. Receiver 500 is configured to receive data from a sensor located inside or outside of a patient's body. The data received by the receiver is utilized to monitor the position and movement of an anatomical structure of the patient. In that regard, the receiver 500 generally receives data representative of a strength of a magnetic field as measured by the sensor. In the illustrated embodiment, the receiver 500 includes a telemetry circuit 502, hardware/software module 504, and display 506. The receiver 500 communicates with a sensor of the present disclosure via wireless telemetry in some instances. In one embodiment, the receiver 500 utilizes its telemetry circuit 502 to facilitate wireless communication with the sensor's telemetry circuit. There are several types of wireless telemetry circuits that are employed for communication between the sensor and receiver 500. For example, RFID, inductive telemetry, acoustic energy, near infrared energy, “Bluetooth,” and computer networks are all suitable means of wireless communication.

Upon receiving data from a sensor, the telemetry circuit 502 of receiver 500 sends the data to hardware/software module 504. The hardware/software module 504 converts or demodulates the data received from the telemetry circuit 502. Furthermore, in some instances the hardware/software module 504 can calculate 3-D images for the position and movement of the anatomical structure being monitored by the sensor. The 3-D images are output to a display 506 where it is displayed in human intelligible form. The conversion and processing of the data can be tailored to the specific liking of the medical personnel in some instances. For example, in some instances the display shows a comparison of the patient's position and movement of the monitored anatomical structure to that of an accepted or established value for an average/normal anatomical structure. These examples of ways in which the data is displayed are for illustration purposes only and in no way limit the ways in which the data can be displayed in accordance with the present disclosure.

The systems of the present disclosure are used to monitor the position and movement of anatomical structures of a patient, including vertebrae of the spinal column in some instances. Specifically, these systems can monitor the flexion, extension, rotation, and/or right and left lateral bending of a patient's spinal column. Furthermore, these systems allow monitoring the position and movement of individual vertebra within the spinal with respect to adjacent vertebrae. Additionally, utilizing the systems of the present disclosure enable monitoring of spinal conditions such as scoliosis, kyphosis, and other degenerative spinal diseases. Further, the systems of the present disclosure are used to monitor the position and movement of anatomical structures such as various joints. For example, the systems of the present disclosure are used to measure the range of motion for a given joint in some instances. The sensors and/or magnetic sources utilized by these systems are affixed to the anatomical structures around a joint and/or the anatomical structures that make up the joint in some instances. In doing so, the movement of a given joint is monitored.

Furthermore, the systems of the present disclosure are used to monitor the growth of short and long bones. Specifically, implanted sensors or magnetic sources are tracked over a time period to determine the relative position of the sensor and/or magnetic source within the bone in order to identify bone growth. Alternatively, two or more sensors or magnetic sources are affixed to the same anatomical structure, so that the spatial relationship between the two or more sensors and/or magnetic source is monitored to determine the amount of bone growth.

Additionally, the systems of the present disclosure are used to monitor the position and movement of anatomical structures such as body organs. As one example, sensors and/or magnetic sources can be affixed adjacent to the heart of a patient. Accordingly, the patient's heart rate and the contraction strength of the heart are monitored by monitoring the movement of the sensors and/or magnetic sources as disclosed herein. Likewise, sensors and/or magnetic sources can be affixed adjacent to the lungs. Attachment of sensors and/or magnetic sources to the lungs enables monitoring of a patient's respiration rate in a similar manner. Furthermore, sensors and/or magnetic sources can be affixed to the gastrointestinal tract to monitor the movements of the pharynx, esophagus, stomach, small intestine, and large intestine.

Further, the sensors and magnetic sources of the present disclosure do not need to have a line-of-sight with respect to each other to function properly. Rather, when either of the sensor or magnetic source is positioned outside of the patient's body it can be placed anteriorly, posteriorly, superior, inferior, and/or laterally of the patient and still function properly. Additionally, the sensors and magnetic sources can be positioned at any angle with respect to the patient's anatomical structures.

Further, the devices, systems, and methods of the present disclosure are utilized to track internal anatomical features of human patients as well as veterinary patients in some instances. In that regard, the size, strength, and/or sensitivity of the sensors and/or magnetic sources is tailored to the amount of tissue and the density of the tissue through which the magnetic fields will pass in some instances. Accordingly, in some embodiments the devices, systems, and methods of the present disclosure are tailored for use in a particular type of patient and/or a particular use in a patient.

While the present invention has been illustrated by the above description of embodiments, and while the embodiments have been described in some detail, it is not the intention of the applicant to restrict or in any way limit the scope of the invention to such detail. Additional advantages and modifications will readily appear to those skilled in the art. Therefore, the invention in its broader aspects is not limited to the specific details, representative apparatus and methods, and illustrative examples shown and described. Accordingly, departures can be made from such details without departing from the spirit or scope of the applicant's general or inventive concept. It is understood that all spatial references, such as “posteriorly,” “anteriorly,” “superior,” “inferior,” and “lateral,” are for illustrative purposes only and can be varied within the scope of the disclosure.

What is claimed:

1. A method of tracking one or more anatomical features of a patient, comprising:
introducing a first implantable magnetic source into a patient's body;
fixedly securing the first implantable magnetic source to a first portion of a first anatomical feature of the patient; and
monitoring a magnetic field generated by the first implantable magnetic source with a magnetic sensing system positioned outside the patient's body in order to track a position of the first implantable magnetic source fixedly secured to the first anatomical feature.

2. The method of claim 1, further comprising:
introducing a second implantable magnetic source into the patient's body;
fixedly securing the second implantable magnetic source to a second portion of the first anatomical feature of the patient spaced from the first portion of the first anatomical feature;
introducing a third implantable magnetic source into the patient's body;
fixedly securing the third implantable magnetic source to a third portion of the first anatomical feature of the patient spaced from the first and second portions of the first anatomical feature; and
monitoring magnetic fields generated by the first, second, and third implantable magnetic sources with the magnetic sensing system positioned outside the patient's body in order to track positions of the first, second, and third implantable magnetic sources fixedly secured to the first anatomical feature.

3. The method of claim 2, further comprising: calibrating the magnetic sensing system positioned outside the body for use with the first, second, and third implantable magnetic sources.

4. The method of claim 3, wherein calibrating the magnetic sensing system for use with the first, second, and third implantable magnetic sources comprises obtaining a baseline measurement of the magnetic fields generated by the first, second, and third implantable magnetic sources at a baseline position.

5. The method of claim 4, wherein monitoring the magnetic fields generated by the first, second, and third implantable magnetic sources in order to track positions of the first, second, and third implantable magnetic sources comprises determining a relative position of the first, second, and third implantable magnetic sources to the baseline position.

6. The method of claim 2, wherein the first anatomical feature is a vertebra;
wherein the first portion of the vertebra is a first transverse process;
wherein the second portion of the vertebra is a second transverse process;
wherein the third portion of the vertebra is a spinous process.

7. The method of claim 6, wherein at least one of the first, second, and third implantable magnetic sources is fixedly secured to the first, second, or third transverse process, respectively, by threadingly engaging a housing of the first, second, or third implantable magnetic source with the first, second, or third transverse process.

8. The method of claim 1, wherein the first implantable magnetic source is a permanent magnet.

9. The method of claim 1, wherein the first implantable magnetic source is an electrical circuit configured to generate a magnetic field.

10. The method of claim 9, wherein the electrical circuit is configured to generate magnetic fields of varying strength.

11. The method of claim 1, further comprising: calibrating the magnetic sensing system positioned outside the body for use with the first implantable magnetic source.

12. The method of claim 11, wherein calibrating the magnetic sensing system for use with the first implantable magnetic source comprises obtaining a baseline measurement of the magnetic field generated by the first implantable magnetic source at a baseline position; and
wherein monitoring the magnetic field generated by the first implantable magnetic source in order to track the position of the first implantable magnetic source comprises determining a relative position of the first implantable magnetic source to the baseline position.

13. A method of tracking anatomical features of a patient, comprising:
introducing a first implantable magnetic sensor into a patient's body;
fixedly securing the first implantable magnetic sensor to a first portion of a first anatomical feature of the patient; and
monitoring a strength of a magnetic field generated by a magnetic source positioned outside the patient's body with the first implantable magnetic sensor in order to track a position of the first implantable magnetic sensor relative to the magnetic source.

14. The method of claim 13, further comprising:
introducing a second implantable magnetic sensor into the patient's body;
fixedly securing the second implantable magnetic sensor to a second portion of the first anatomical feature of the patient spaced from the first portion of the first anatomical feature;
introducing a third implantable magnetic sensor into the patient's body;
fixedly securing the third implantable magnetic sensor to a third portion of the first anatomical feature of the patient spaced from the first and second portions of the first anatomical feature; and
monitoring the strength of the magnetic field generated by the magnetic source positioned outside the patient's body with the first, second, and third implantable magnetic sensors in order to track positions of the first, second, and third implantable magnetic sensors relative to the magnetic source.

15. The method of claim 14, further comprising: calibrating the first, second, and third implantable magnetic sensors for use with the magnetic source.

16. The method of claim 15, wherein calibrating the first, second, and third implantable magnetic sensors comprises obtaining a baseline measurement of the strength of the magnetic field generated by the magnetic source at a baseline position.

17. The method of claim 16, wherein monitoring the strength of the magnetic field generated by the magnetic source in order to track positions of the first, second, and third implantable magnetic sensors comprises determining a relative position of the first, second, and third implantable magnetic sensors to the baseline position.

18. The method of claim 13, wherein the first anatomical feature is a vertebra;
wherein the first portion of the vertebra is a first transverse process;
wherein the second portion of the vertebra is a second transverse process; and
wherein the third portion of the vertebra is a spinous process.

19. The method of claim 18, wherein at least one of the first, second, and third implantable magnetic sensors is fixedly secured to the first, second, or third transverse process, respectively, by threadingly engaging a housing of the first, second, or third implantable magnetic sensor with the first, second, or third transverse process.

20. A method comprising:
gaining minimally invasive access to a first bony anatomical feature;
introducing at least one implantable device containing a permanent magnet into a patient's body through the minimally invasive access;
threadingly engaging a housing of the at least one implantable device with the first bony anatomical feature through the minimally invasive access;
closing the minimally invasive access to the first bony anatomical feature with the at least one implantable device threadingly engaged with the first bony anatomical feature; and
monitoring a magnetic field generated by the permanent magnet contained by the at least one implantable device with a magnetic sensing system positioned outside the patient's body in order to track a position of the at least one implantable device threadingly engaged with the first bony anatomical feature.
